(19) United States
(12) Patent Application Publication
(10) Pub. No.: US 2024/0038092 A1
(43) Pub. Date: Feb. 1, 2024

(54) SYSTEM AND METHOD FOR ENABLING A USER TO OVERCOME A MALADAPTIVE PERSONALITY TRAIT

(71) Applicant: DHARMA LIFE SCIENCES LLC, Sleepy Hollow, NY (US)

(72) Inventors: JONNALAGADDA SASIDHAR, Sleepy Hollow, NY (US); Sreenidhi SUNDARAN, Bangalore (IN); Shanthi Lakshmi DURAIMANI, Bangalore (IN)

(21) Appl. No.: 18/115,751

(22) Filed: Feb. 28, 2023

Related U.S. Application Data
(63) Continuation of application No. 16/054,893, filed on Aug. 3, 2018, now abandoned.
Application No. 16/054,893 is a continuation-in-part of application No. 15/002,417, filed on Jan. 21, 2016, now abandoned.
Application No. 15/002,417 is a continuation-in-part of application No. 14/931,868, filed on Nov. 4, 2015, now abandoned.
Application No. 16/054,893 is a continuation-in-part of application No. 14/975,885, filed on Dec. 21, 2015, now abandoned.
Application No. 14/975,885 is a continuation-in-part of application No. 14/975,888, filed on Dec. 21, 2015, now abandoned.
Application No. 14/975,888 is a continuation-in-part of application No. 14/975,881, filed on Dec. 21, 2015, now abandoned.
Application No. 14/975,881 is a continuation-in-part of application No. 14/985,472, filed on Dec. 31, 2015, now abandoned.
Application No. 14/985,472 is a continuation-in-part of application No. 14/985,475, filed on Dec. 31, 2015, now abandoned.
Application No. 14/985,475 is a continuation-in-part of application No. 14/985,478, filed on Dec. 31, 2015, now abandoned.
Application No. 14/985,478 is a continuation-in-part of application No. 15/002,416, filed on Jan. 21, 2016, now abandoned.
Application No. 15/002,416 is a continuation-in-part of application No. 15/002,418, filed on Jan. 21, 2016, now abandoned.
Application No. 15/002,418 is a continuation-in-part of application No. 15/077,946, filed on Mar. 23, 2016, now abandoned.
Application No. 15/077,946 is a continuation-in-part of application No. 15/182,633, filed on Jun. 15, 2016, now abandoned.
Application No. 15/182,633 is a continuation-in-part of application No. 15/213,423, filed on Jul. 19, 2016, now abandoned.
Application No. 15/213,423 is a continuation-in-part of application No. 15/455,159, filed on Mar. 10, 2017, now abandoned.

(51) Int. Cl. G09B 19/00; G16H 20/70; G16H 50/20; G16H 50/30; G16H 20/30; G16H 20/60

(52) U.S. Cl. CPC G09B 19/00; G16H 20/70; G16H 50/20; G16H 50/30; G16H 20/30; G16H 20/60; G09B 5/02

(57) ABSTRACT

A system is provided for enabling a user to overcome anger. The system is configured to enable the user to engage in a first and a second activity in a virtual environment, directed to rewire a first and a second defective wiring, respectively. The first defective wiring results in an individual's bias towards noticing unfair attributes in a situation or an event. The second defective wiring results in inability to weigh between fair and unfair attributes of a situation. Symptom of the first and the second defective wirings is anger. Engagement in the second activity is enabled after the user has at least participated in the first activity. Automated instructions are provided to the user to engage in activities performed in a real world environment directed to rewire the first and the second defective wiring.

### CROSS REFERENCE TO RELATED APPLICATION

This application is a continuation-in-part application of U.S. patent application Ser. No. 14/931,868 filed Nov. 4, 2015 entitled SYSTEM AND METHOD FOR ENABLING A USER TO OVERCOME WEAK BEHAVIORAL TRAITS, which is hereby incorporated herein by reference. Benefits of priorities to all related applications are claimed.

### BACKGROUND

Unless otherwise indicated herein, the materials described in this section are not prior art to the claims in this application and are not admitted to be prior art by inclusion in this section.

The subject matter in general relates to behavioral traits in humans, and more particularly but not exclusively, the subject matter is directed to a technical solution to overcome anger, which is a weak behavioral trait.

An individual's behavior towards others, attitudes and characteristics, defines his personality. Each individual's personality is a combination of both positive and negative traits. A person may be driven by his positive traits at times and by negative traits at other times. As a result, to classify a person into a single personality type is difficult, as there are several different behavioral traits a person can have. Behavioral traits may be influenced by genes and/or by the environment where he grew up or ma have spent most of his time during the stage of brain development. As an example, anger, in an individual, may be caused by genetic predisposition, the environment in which the individual was raised where anger is a way of communication, discrimination of an individual by caregivers as compared to his siblings, physical, sexual and verbal abuse and tendency to bottle up feelings till defenses become low, among others.

Understanding what makes people who they are has been a challenge in the world of psychology. One of the approaches used for determining the personality of a person is by way of taking a psychology based personality test. In such a test, the person answers a set of questions, and based on the answers gets a report detailing the type of personality that person may have. On the other hand, behavior of a person may be observed to determine weak personality traits in him. Behavior of a person with anger may include solely focusing on what he needs and not thinking about others concerns, tendency to blame others rather than taking responsibility for the consequences of their anger, assertion of their own opinion as fact and finding others' opinions irrelevant, resort to undermine others to make them self look better by comparison, among others.

Negative or weak traits in a person can lead to difficulty in achieving their life goals. Conventionally, people try to overcome their weaknesses when one understands one's weaknesses or weak traits. An individual may approach a counselor or a psychologist with a view that, the counselor or the psychologist may be able to help him address his negative or weak trait. The person may have to undergo several sessions with the counselor. Such sessions may deal with the negative trait via a broad based general (not focused at the root of the weak trait) approach, which may be effective in the short run. For example, if a person is trying to deal with stress, the person may be advised to engage in regular exercise, outdoor games, engage socially and so on, which may prove effective to an extent the time being.

Currently, there are multiple games and applications that may work in the same way as a counseling process. Even these games deal with the negative trait via a general approach. As an example if a person is not capable of reading at good speed, the person is subjected to games related to reading to improve his speed of reading. Such approach may be effective in the short run and does not address negative traits in a wholesome manner

In light of the foregoing discussion there is a need for an improved technique to overcome anger, which can be classified as a weak personality trait based on the goals of the person.

### SUMMARY

An embodiment provides a system for enabling a user to overcome anger. The system includes an activity module. The activity module is configured to enable the user to engage in at least a first activity in a virtual environment. The first activity is directed to rewire a first defective wiring of the brain. The first defective wiring results in an individual's bias towards noticing unfair attributes in a situation or an event. The activity module is further configured to enable the user to engage in at least a second activity in the virtual environment. The second activity is directed to rewire a second defective wiring of the brain. The second defective wiring results in inability to weigh between fair and unfair attributes of a situation. Symptom of the first and the second defective brain wirings is anger. Additionally, the activity module is configured to provide automated instructions to the user to engage in at least one activity, wherein the one activity is performed in a real world environment. The activity is directed to rewire the first defective wiring of the brain. Furthermore, the activity module is configured to provide automated instructions to the user to engage in yet another activity. The yet another activity is performed in the real world environment. The yet another activity is directed to rewire the second defective wiring of the brain.

Another embodiment provides a method for enabling a user to overcome anger. The method includes enabling the user to engage in at least a first activity in a virtual environment. The first activity is directed to rewire a first defective wiring of the brain. The first defective wiring results in bias or inclination towards noticing unfair attributes in a situation or an event. The user is enabled to engage in at least a second activity in the virtual environment. The second activity is directed to rewire a second defective wiring of the brain. The second defective wiring results in inability to weigh between fair and unfair attributes of a situation. Symptom of the first and the second defective wirings is anger. Further, automated instructions are provided to the user to engage in at least one activity. The one activity is performed in a real world environment. The activity is directed to rewire the first defective wiring of the brain. Furthermore, automated instructions are provided to the user to engage in yet another activity. The yet another activity is performed in the real world environment. The vet another activity is directed to rewire the second detective wiring of the brain.

Yet another embodiment provides a system for enabling a user to overcome anger. The system includes an activity module configured to enable the user to engage in at least a first activity in a virtual environment, wherein the first activity is directed to rewire a first defective wiring of the brain, wherein the first defective wiring results in an individual's bias towards noticing unfair attributes in a situation or an event. The activity module is further configured to enable the user to engage in at least a second activity in the virtual environment, wherein the second activity is directed to rewire a second defective wiring of the brain, wherein the second defective wiring results in inability to weigh between fair and unfair attributes of a situation, wherein symptom of the first and the second defective wirings is anger, wherein engagement in the second activity is enabled after the user has at least participated in the first activity.

### BRIEF DESCRIPTION OF THE DRAWINGS

Embodiments are illustrated by way of example and not limitation in the Figures of the accompanying drawings, in which like references indicate similar elements and in which:

FIG. 1 is an exemplary architecture of an exemplary system 100 to overcome anger;

FIG. 2 is a block diagram of an exemplary activity module 200 to overcome anger;

FIG. 2A is a block diagram of an exemplary behavioral traits database 202 of the activity module 200 to overcome anger;

FIG. 2B is a block diagram of an exemplary collection of games 204 present in the activity module 200 to overcome anger;

FIG. 2C is a block diagram of an exemplary actions database 206 of the activity module 200 to overcome anger; and

FIG. 3 illustrates a flowchart of an exemplary method for overcoming anger.

### DETAILED DESCRIPTION

I. OVERVIEW
II. PRINCIPLE
III. SYSTEM ARCHITECTURE
IV MODULES OF SYSTEM TO OVERCOME ANGER
V. METHOD FOR OVERCOMING ANGER

The following detailed description includes references to the accompanying drawings, which form part of the detailed description. The drawings show illustrations in accordance with example embodiments. These example embodiments are described in enough detail to enable those skilled in the art to practice the present subject matter. However, it will be apparent to one of ordinary skill in the art that the present invention may be practiced without these specific details. In other instances, well-known methods, procedures and components have not been described in detail so as not to unnecessarily obscure aspects of the embodiments. The embodiments can be combined, other embodiments can be utilized or structural and logical changes can be made without departing from the scope of the invention. The following detailed description is, therefore, not to be taken as a limiting sense.

In this document, the terms “a” or “an” are used, as is common in patent documents, to include one or more than one. In this document, the term “or” is used to refer to a nonexclusive “or,” such that “A or B” includes “A but not B,” “B but not A,” and “A and B,” unless otherwise indicated.

### I. Overview

Embodiments provide a technical solution to overcome anger, which is a weak behavioral trait. The solution is based on a principle that genetic and environmental factors (may be referred to as “cause”) cause defects in human brain wiring (may be referred to as “defect”), and such defects in human brain wiring express themselves in the form of anger (may be referred to as “symptoms”); hence, to overcome anger, the defective brain wirings have to be rewired, thereby providing a wholesome and long term solution to weak behavioral traits.

In an embodiment, a system is provided to overcome anger. The system may include an activity module. The activity module may be configured to enable the user to engage in activities in a virtual environment. The activities in the virtual environment facilitate rewiring of defective brain wirings, wherein the symptom of the defective brain wirings is anger. The activity module may be further configured to provide automated instructions to gradually expose the user to situations in a real environment. The situations may be generally uncomfortable to the user as a result of the defective brain wirings. Exposure to such situation facilitates rewiring of the defective brain wirings.

The activity in the virtual environment may be a brain game. There may be a plurality of levels in the brain game. Each level in the game may have to be successfully completed by the user to unlock a subsequent higher level in the game.

The real world activity may take place in the real world environment, subsequent to the brain games. There may be a plurality of levels in the real world activity.

### II. Principle

Anger is a very powerful basic human emotion that can stem from feelings of frustration, hurt, annoyance or disappointment. It makes an individual hostile and unpleasant because of something unfair or unkind that has happened.

Anger is in fact a symptom or consequence of the way an individual's brain is wired. The way an individual's brain is wired is based on the joint influence of genetic and environmental factors. With regards to each kind of brain wiring, genetic and environmental factors may work together in different proportions.

With regards to anger, the brain of the person with anger is wired such that the person tends to be biased to notice unfair attributes in a situation or an event, which is a defective brain wiring. Additionally, the brain of the person with anger is wired such that the individual is unable to weigh between fair and unfair attributes of a situation, which may be considered as another defective brain wiring. Such defective brain wiring may have been caused by one or more of genetic predisposition, the environment in which the individual was raised where anger has been a way of communication, discrimination of an individual by caregivers as compared to his siblings, physical, sexual and verbal abuse and tendency to bottle up feelings trill defenses become low, among others.

Embodiments provide a solution to rewire the defective brain wirings (addressing the defect) rather than just addressing the behavioral trait (symptom) without addressing the core defect. The solution is directed towards rewiring the defective brain wirings such that the ability to identify the fair attributes of a situation is increased and ability to weigh between fair and unfair attributes of a situation is increased.

It is important to identify the brain wiring that results in development of a trait. A wholesome approach to overcome or eradicate a negative or a weak behavioral trait in a person would be to implement ways to rectify defect in the brain wiring.

Brain wirings may be referred to as connections of neural pathways in the brain. These connections of the brain form beliefs, thought patterns and response patterns to external stimulation. Rewiring may be referred to as a process of creating new brain wiring and stimulating a person's existing brain wirings so that his beliefs, thought patterns and responses to external stimulation change for the better.

Individuals with anger tend to be biased to notice unfair attributes in a situation or an event and they fail to weigh between fair and unfair attributes of a situation. Different brain regions and neural network of the individual are responsible for such defects. Amygdala (AMY), Hypothalamus (HYP), Periaqueductal Grey (PGY) and the Frontal Cortex (FC) are important brain regions or blocks. When an angry person experiences internal or external stimuli, simultaneously amygdala, hypothalamus, and periaqueductal grey get activated. These regions are regulated by several regions in the frontal cortex, such as orbito frontal cortex, ventro medial, ventro lateral cortex, dorso medial, and dorso lateral frontal cortex. These regions send connection to inhibitor inter neurons within amygdala leading to decrease in amygdala activation, which eventually decrease the activity of hypothalamus, and periaqueductal grey. However, in angry people, the connection between frontal cortex and amygdala is less activated.

### III. System Architecture

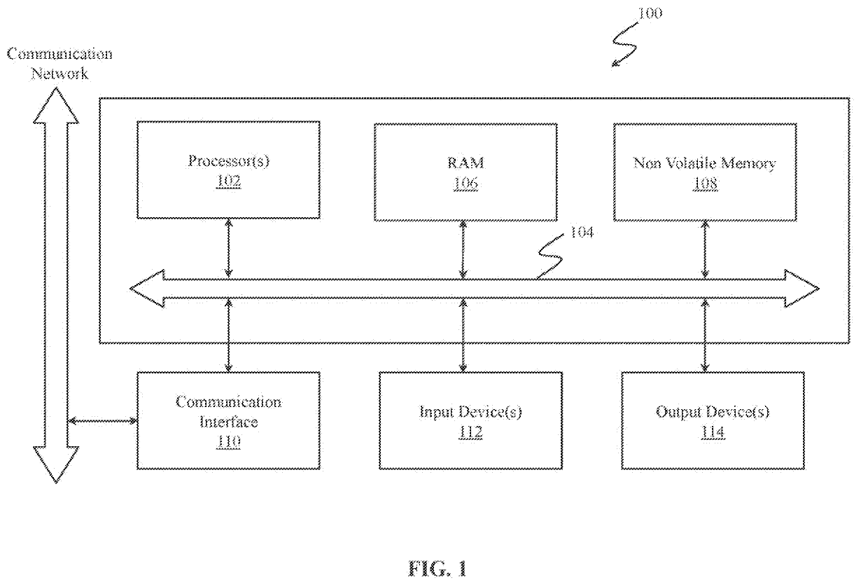

Referring to the figures, more particularly to FIG. 1, an exemplary architecture of an exemplary system 100 to overcome anger is provided. The system 100 include one or more processors 102, a bus system 104, a random access memory 106, a disk drive or non-volatile memory 108, a communication interface 110 input device(s) 112 and output device(s) 114. Further, functionality of the system 100 may be distributed across multiple devices that may be located remotely from each other.

The processor 102 may be any hardware which returns output by accepting signals, such as electrical signals as input. In one embodiment, processors 102 may include one or more processing units (CPUs). The processor(s) 102 may communicate with a number of peripheral devices via the bus system 104. The processor(s) 102 may be implemented as appropriate in hardware, computer-executable instructions, firmware, or combinations thereof. Computer-executable instruction or firmware implementations of the processor(s) 102 may include computer-executable or machine-executable instructions written in an suitable programming language to perform the various functions described.

Communications interface 110 may provide an interface to other communication networks and devices. The input devices 112 may include all possible types of devices and mechanisms for inputting information to system 100. The output devices 114 may include all possible types of devices and mechanisms for outputting information from the system 100. The system 100 may include memory that may store data and program instructions that are loadable and executable on the processor(s) 102, as well as data generated during the execution of these programs. The memory may be volatile, such as random access memory 106 and/or a disk drive or non-volatile memory 108.

### IV. Modules of System to Overcome Anger

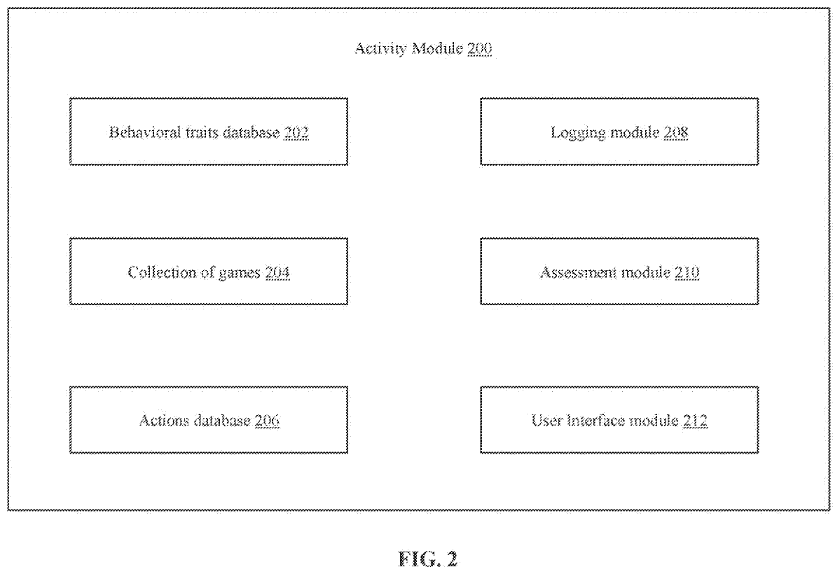

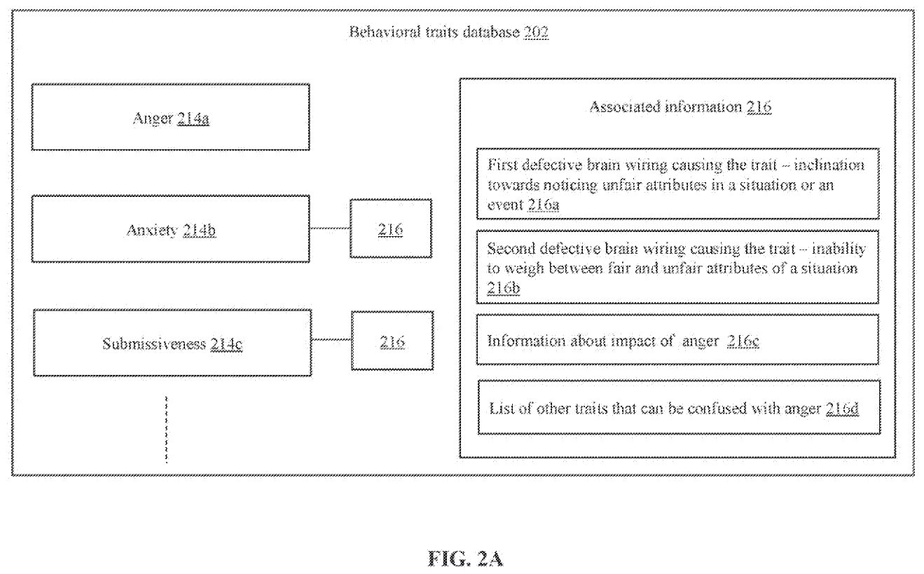

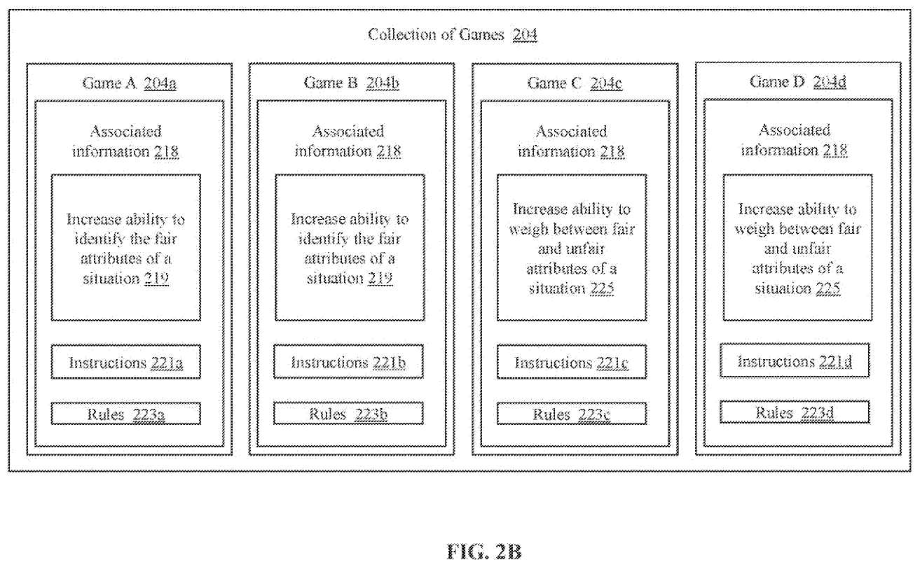

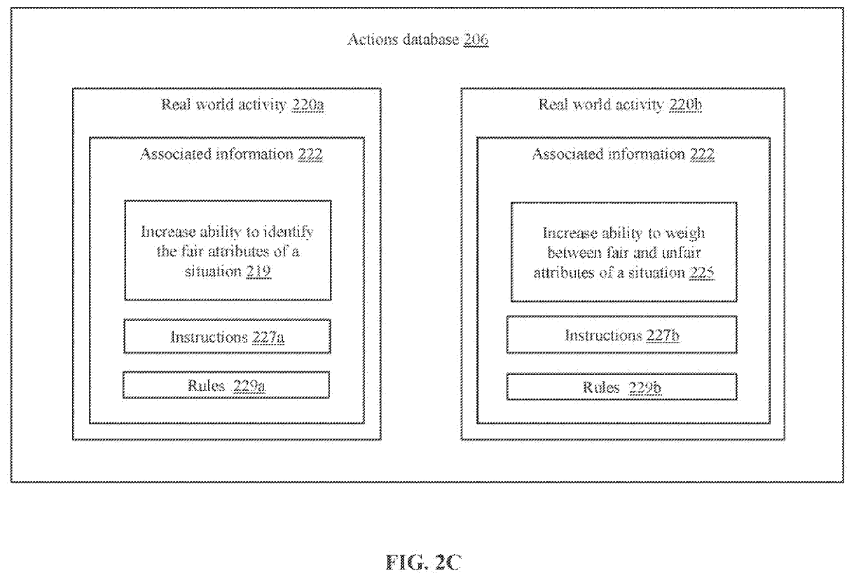

Referring to the figures, and more particularly to FIG. 2, the system 100 includes various modules for enabling users to overcome anger. The system 100 includes an activity module 200. The activity module 200 may be a gaming module or an application module. The activity module 200 may be hosted by a communication device that enables a user to play games. Examples of such communication devices may include smart phone, tablet, notebook, laptop and desktop computer, among other devices.

The activity module 200 may include a behavioral traits database 202, a collection of games 204, an actions database 206, a logging module 208, an assessment module 210 and a user interface module 212.

### Behavioral Traits Database 202

Referring to FIG. 2A, in an embodiment, the behavioral traits database 202 may include a list of behavioral traits 214. Examples of behavioral traits 214 include, but not limited to, anger 214a, anxiety 214b and submissiveness 214c, among others. The traits database 202 may further include, with respect to one or more of the behavioral traits, information associate 216 with the trait 214.

With regards to anger 214a, the associated information 216 may include information corresponding to the defective brain wirings (defects) 216a and 216b causing anger (symptom of the defect), information about impact 216c of anger 214a on a person, and a list 216d comprising one or more traits that may be confused with anger 214a, among other information. Some or all of the associated information 216 may be displayed to the user of the activity module 200.

A first detective brain wiring 216a causing anger 214a is the brain wiring causing bias to notice unfair attributes in a situation or an event. A second defective brain wiring 216b causing anger 214a is the brain wiring causing an individual to fail to weigh between fair and unfair attributes of a situation.

In an embodiment, the information corresponding to the first and second defective brain wiring 216a, 216b causing anger 214a enables the activity module 200 to select games, activities or actions to overcome anger 214a.

In an embodiment, displaying the information corresponding to the defective brain wiring (defect) 216a, 216b causing anger 214a helps the user in understanding the defects responsible for anger 214a, thereby motivating the user to follow the instructions provided by the activity module 200.

Information about impact 216c of anger 214a can include one or more of solely focusing on what an individual with auger needs and not thinking about others concerns, tendency to blame others rather than taking responsibility for the consequences of their anger, assertion of their own opinion as fact and finding others' opinions irrelevant, resort to undermine others to make them self look better by comparison, among others.

The list 216d of other traits that can be confused with anger 214a include impatience, tack of self control and low empathy, among others.

In an embodiment, the activity module 200 displays the information corresponding to the brain rewiring that the activity module 200 is attempting to achieve to overcome the weak behavioral trait 214, thereby enabling the user to understand the actual goal he will be instructed to work towards, which in turn can overcome anger 214a.

With regards to anger 214a, the brain rewiring attempted to achieve to overcome anger 214a includes rewiring the detective brain wirings 216a, 216b such that ability to identify the fair attributes of a situation is increased and ability to weigh between fair and unfair attributes of a situation is increased.

In an embodiment, the activity module 200 may display the information corresponding to the causes (genetic or environmental) that resulted in the defective brain wirings (defect) 216a, 216b, which in turn resulted in anger 214a, thereby enabling the user to relate to the cause, defect and symptom of the defect, which results in a wholesome experience while working to overcome anger 214a.

### Collection of Games 204

The activity module 200 includes a collection of digital games 204, which are played in a virtual world. Each of the games 204 is used by the activity module 200 based on the brain rewiring, which the game 204 is capable of achieving.

Referring to FIG. 2B, in an embodiment, each game 204a-204d (may be referred to as game 204 or games 204) may include associated information 218. As an example, a game may be capable of achieving more than one type of brain rewiring. Alternatively, one single type of rewiring may be achieved through more than one game. Each game 204 includes data indicating the brain rewiring(s) 219, 225 it is capable of achieving, so that the activity module 200 can opt to present a choice of one or more games, which has the capability to achieve the brain rewiring, which the activity module 200 is attempting to achieve.

The associated information 218 may further include instructions 221 to be followed by the user to play the game 204. The associated information 218 may additionally include one or more rules 223. Examples of rules include rules for playing each game, rules to proceed to a higher level, scoring method in each level and time assigned to each level to reach the end of the level, among others.

In an embodiment, a higher level of the digital game 204 is unlocked based on predefined criteria.

In an embodiment, the higher level is unlocked upon repeatedly playing a previous level in the digital game 204 for a predefined number of times or duration of time.

In the example presented in FIG. 2B, four different games 204a-204d are included in the collection of games 204. The four games 204a, 204b, 204c and 204d can be used by the activity module 200 to correct the defective brain wirings 216a and 216b by rewiring the brain to increase the ability to identify the fair attributes of a situation 219 and increase ability to weigh between fair and unfair attributes of a situation 225.

Game A 204a and Game B 204b are directed at rewiring 219 the brain to increase the ability to identify the fair attributes of a situation. The games, 204c and 204d are directed at rewiring 225 the brain to increase the ability to weigh between fair and unfair attributes of a situation.

Game A 204a may be designed to rewire the brain to increase the ability to identify the fair attributes of a situation 219. Game A 204a may be developed to train individuals with anger to reduce their bias towards noticing the unfair attributes of a situation or an event. Individuals with anger issues have a tendency to focus on threatening/angry faces in the environment. They also tend to take more time than a healthy individual to interpret what emotion the other person is going through. Selective bias training focuses on cues such as faces in social environments (both threatening and non threatening). Game A 204a may be helpful in training individuals to focus on the non threatening faces. The game would also train them to interpret the appropriate emotions that other people go through faster and focus on the non threatening faces among different faces in social environments (both threatening and non threatening). The goal may be to see how fast they select the positive stimuli over the threatening stimuli. A track of the time ma be kept but may not be shown to the user. The instruction 221a may include recommendation to play the game 204a for a minimum duration every day. The rules 223a may allow proceeding to a next level in the game 204a once a desired level of performance is achieved, for example, once they are able to select the positive stimuli in less than 100 ms for at least ten pairs in each level, only then they can be allowed to move to the next level. The activity module 200 may be configured to define or set complexities for one or more digital games. Complexities may be numbered on a scale of 1 to 10.

At complexity 1, two stimuli, one happy and one threatening face of real people will be shown on a screen. The user has to select the happy face over the threatening thee. At complexity 2, stimuli of faces will be either happy and neutral or neutral and threatening faces. At the first instance, the happy face has to be selected and at the second instance, the neutral face has to be selected. At complexity 4, four stimuli (could be happy or threatening faces of real people) will be shown on the screen. These stimuli (but with different faces) will repeat (1 happy and 3 threatening). The user has to select the happy faces over the threatening faces. At complexity 4, four stimuli, (1 happy, 2 threatening and 1 neutral or 1 happy, 2 neutral and 1 threatening) will be shown to the individual. The individual has to select the happy faces in all cases

Game B 204b may be designed to increase one's ability to identify the fair attributes of a situation 219. The instruction 221b may include recommendation to play the game 204b for at least a predefined duration for predefined number of days. The rules 223b may allow proceeding to a next level in the game 204b once a desired level of performance is achieved. The activity module 200 may be configured to define a complexity 7 for this game. The number of stimuli presented for this game may be 25.

In the game 204b, the action or behavior of another person towards the individual will be represented in a word. Instructions (221b) may be provided to choose a word from a list that explains the behavior positively. If the interpretation is incorrect, the individual has to rethink and answer once again 1. The list may be presented in Table 1.

| | | |
|---|---|---|
| Hiding the truth | Cheating | White Lies |
| | Forgery | Exaggeration |
| | Deceit | Pranking |
| | Fake | |
| | Dishonest | |
| | Fabricate | |
| | Deception | |
| Making Fun | Hurting | Kidding |
| | Rude | Amusing |
| | Teasing | Enjoyable |
| | Bullying | Lively |
| | Humiliating | Friendly |
| Mistake | Corrupt | Unplanned |
| | Immoral | Environment |
| | Lawless | Upbringing |
| | Evil | |
| | Misconduct | |
| | Fault | |
| | Wrong | |
| | Atrocity | |
| Negative Remarks | Disrespect | Constructive Criticism |
| | Disgrace | Opinion |
| | Abuse | Appraisal |
| | Rude | Motivation |
| | Unpleasant | |
| | Shame | |
| | Outrage | |
| Embarrass | Disturbing | Constructive Criticism |
| | Annoying | Joking |
| | Shame | Kidding |
| | Rude | |
| | Disrespect | |
| | Disgrace | |
| Ignored | Neglect | Busy |
| | Reject | Working |
| | Avoid | Occupied |
| | | Engaged |
| Sharp Personality | Rude | Unintentional |
| | Spiteful | Smart |
| | Unkind | Straight forward |
| | Unfriendly | Stoic |
| Nagging | Annoying | Motivating |
| | Irritating | Persuasive |
| | Troublesome | Persistent |
| | Clingy | Assertive |
| Undiplomatic | Disrespect | Indifferent |
| | Insult | Nonaligned |
| | Impolite | Preoccupied |
| | Ill-mannered | Straight forward |
| Inadequacy | Failure | Unintentional |
| | Blunder | Genuine |
| | Mishap | Helplessness |
| | Disappointing | |
| Nontransparent | Deceiving | Diplomatic |
| Personality | Dishonesty | Defensive |
| | Fraud | Holding Back |
| | Fake | Fear of Judgment |
| | | Comfort Zone |
| | | Trying to impress |
| Differential | Partial | Motivating |
| Treatment | Prejudiced | Unintentional |
| | Discriminating | Circumstances |
| | Animosity | Obligation |
| | Unfair | |
| | Biased | |
| Breaking Rules | Unauthorized | Helplessness |
| | Unlawful | Upbringing |
| | Illegitimate | Uninformed |
| | Prohibited | Circumstances |
| | Wrong | |
| | Immoral | |
| | Atrocity | |
| | Irresponsible | |
| Against Social | Wrong | Circumstances |
| Morals | Valueless | Cultural difference |
| | Selfish | Emotional/Heat of the |
| | Low life | moment |
| | Emotionless | Peer Pressure |
| | | Unavoidable |
| | | Disorder |
| stealing | Crime | Poverty |
| | Horrible | Bad Company |
| | Cheap | Circumstances |
| | Immoral | Upbringing |
| | Selfish | Compulsive Disorder |
| | Valueless | Took by mistake |
| Poor performance | Incapable | Nervousness |
| | Lazy | Personal problems |
| | Unprepared | Issues |
| Less knowledgeable | Ignorance | Modest |
| | Unintelligent | Lack of exposure |
| | Dumb | Potential |
| | Shallow | |
| Somebody lost your | Misplacement | Helpless |
| objects | Carelessness | Unintentional |
| | Irresponsible | Absent minded |
| | Insensitive | |
| Interruption | Disturbance | Important |
| | Annoying | Emergency |
| | Inconsiderate | Informative |
| | Manner less | Unintentional |
| | | Friendship |
| Conservative | Narrow minded | Experienced |
| | Closed minded | Generation-gap |
| | Restrictive | Principled |
| | | Lack of Exposure |
| Correction | Fault finding | Caring |
| | Dominating | Helping |
| | Negative | Well wisher |
| | Picking on | Improvement |
| | | Perfectionist |
| Argument | Arrogant | Opinion |
| | Threatening | Sorting out |
| | Rude | Making a point |
| | Insensitive | |
| | Dominating | |
| | Stubborn | |
| Force | Manipulative | Well wisher |
| | Selfish | Motivating |
| | Dominating | In need |
| | Restrictive | Obligation |
| | Using | |
| Unequal Treatment | Unfair | Unintentional |
| | Biased | Practicality |
| | Discriminating | Obligation |
| | Partial | Cultural difference |
| | Purposeful | |
| | Mean | |
| | Rude | |
| Talking in your | Betrayal | Casual comment |
| absence | Two-faced | Unintentional |
| | Slanderer | |
| | Backbiting | |
| Teasing | Making fun | Kidding |
| | Bullying | Friendly |
| | Insulting | Funny |
| | Mean | Bonding |
| Criticism | Putting down | Constructive |
| | Fault finding | Helping |
| | Mean | Straight forward |
| | Insult | Motivating |
| | | Well meaning |
| Disobeying | Rude | Obligation |
| | Audacity | Circumstances |
| | Ill mannered | Inability |
| | Liar | Different opinion |
| | Arrogance | |
| | Disloyal | |
| Expectation not | Lazy | Unintentional |
| Matched | Carelessness | High expectation |
| | Not bothered | Helpless |
| | Taking for Granted | Difference in |
| | Inadequate | Preferences |

Game C 204c game may be designed to enhance one's ability to weigh between fair and unfair attributes of a situation 225. The instruction 221c may include recommendation to play the game 204c for at least a predefined duration for a predefined number of days. The rules 223c may allow proceeding to a next level in the game 204c once a desired level of performance is achieved. The activity module 200 may define a complexity 9 for this game and the number of stimuli shown to the individual may be 9.

In the game C 204c, the user may be presented with situations/events and a plurality of facts (selected from the list below) associated with the situation or event. Based on evaluation and analysis of the situation and the facts, the user may have to provide or select the appropriate answers. An example is presented below:
Situation—You did not get through an interview
Fair
   You have another job offer.
   The job is not in your area of expertise.
   Your qualifications can get you a better job.
   This job requires more work experience.
   There were other suitable candidates.
   The job needed someone who could work night shift.
   You applied only for the experience of attending an interview.
   This was a good learning experience
   You have the interview for your dream job coming up
   It allowed you to reflect on your weaknesses and strengths.
Unfair
   You politely disagreed to some of the comments made by the interviewers.
   The interviewers were a little rude
   The moderators didn't let you make your point in the group discussion.
   The company is a competitor of your previous organization.
   The selected candidates were referrals.
   The interviewers expected you to compromise on your remuneration.
   The interviewers made personal comments.
   The interviewers did not give a reason for rejection
   You have to depend on your parents for a living
   The interviewers rejected you because of your age
Should you get angry for being rejected? Yes or No

Game D 204d game may be designed to enhance one's ability to weigh between fair and unfair attributes of a situation 225. The instruction 221d may include recommendation to play the game 204d for at least a predefined duration for a predefined number of days. The rules 223d may allow proceeding to a next level in the game 204d once a desired level of performance is achieved.

In the game D 204d, the user may be presented with situations/events and a plurality of facts associated with the situation or event. Based on evaluation and analysis of the situation and the facts, the user may have to provide or select the appropriate answers. An example is provided below.

### Situation/Event:

Your mother is telling you to put your shoes in the rack. But you have just returned from work all tired, so you just want to go back to our room. Your mother is insisting on putting it back on the rack.
Q. So in this situation, what would you decide?
A:
   You would put it back on the rack and go back to your room.
   You would shout at your mother telling you are very tired.
      You would just walk away.

### Situation/Event:

Then your mother mentions that if you leave the shoes that a you would cause for someone to trip and fall, or the dog might bite them.
Q. So in this situation, what would you decide?
A:
   You would put it back on the rack and no back to cur room.
   You would shout at your mother telling you are tired.
   You would just walk away

### Situation/Event:

Then your mother mentions that if you leave the shoes that way you would cause for someone to trip and fall, or the dog might bite them
Q. So in this situation, what would you decide?
A:
   You would put it back on the rack and go back to your room.
   You would shout at your mother telling you are very tired.
   You would just walk away

### Situation/Event:

Your mother offers a glass of water and asks you to keep the shoes back on the rack.
Q. So in this situation, what would you decide?
A.
   You shout at your mother to keep the shoes on the rack by herself.
   You would put it back on the rack and go back to your room.
   You would shout at your mother by throwing the glass.
   Your mother is trying to discipline you.

### Situation/Event:

When you plan to go out that evening, von find that one of your pair of shoes is dirty because of your brother's shoes.
Q. So in this situation, what would you decide?
A:
   You shout at your brother for keeping the shoes above yours.
   You realize your mistake of not listening to your mother.
   You understand why your mother insisted on putting it hack on the rack.
   You make an issue out of the whole situation and still not realize your mistake.

### Actions Database 206

The actions database 206 includes information corresponding to a plurality of real world activities or sets of real world activities. Real world activities may include activities that are performed in a real world environment, as opposed to the digital games played in the virtual world (ex: games included in the collection of games 204).

Referring to FIG. 2 each of the real world activities 220a-220b (may be referred by numeral 220) or sets of real world activities 220a-220b (may be referred by numeral 220) is used by the activity module 200 based on the brain rewiring 219, 225, which the real world activity 220 is capable of achieving.

Each real world activity 220 may include associated information 222. As an example, a real world activity may be capable of achieving more than one type of brain rewiring. Each real world activity 220 includes data indicating the brain rewiring(s) 219, 225 it is capable of achieving, so that the activity module 200 can opt to present a choice of one or more real world activities 220, which has the capability to achieve the brain rewiring, which the activity module 200 is attempting to achieve.

The associated information 222 may further include instructions 227 to be followed by the user to perform the real world activity 220 or a set of real world activities 220. The associated information 222 may additionally include one or more rules 229. Examples of rules include rules to proceed to a higher level, scoring method in each level and time assigned to each level to reach the end of the level, among others.

The real world activity 220a may be presented to the user after the user has at least participated in one or more games (204a and/or 204b) to a predefined extent that attempts to rewire 219 the first defective brain wiring 216a. The real world activity 220a may be designed to rewire the brain to increase the ability to identify the fair attributes of a situation. Instructions 227a may include recommendation that the action has to be carried out every day and rules 229a may state that each level has to be repeated 5 times, before the next level unlocks.

In a first level, the activity module 200 may provide instructions 227a to choose a quiet place to sit in and close the eyes and imagine in detail what is shown. The activity module 200 may present a list of tasks in a sequence.
Screen 1 shows the tasks:
   Imagine someone/some situation provoke your anger, hold the feeling for few minutes, and perform deep breathing for 5 minutes. Think and come up with one fair attribute. Reflect on the reason for 5 minutes.
Example: Your spouse yelled at you soon after he got back from the office for not keeping the house clean.

### Fair Attributes:

The spouse might have had a bad day at office.
Imagine someone/some situation that provokes your anger, hold the feeling for few minutes, and walk over to a window Where there is sunlight. Think and come up with one fair attributes. Reflect, on the reason for 5 minutes.
Situation: My boss criticized my work.
Feelings:
   How can he criticize my work.
   I am very hard working, and work more than 9 hours a day,
   I am working for this company for a long time, and have lot of experience in handing work.
   He's very rude, mean.

### Fair Attributes:

I might have made some errors. It is obvious that it is my mistake. So it is fair from his side to get angry.
Imagine someone/some situation provoke your anger, hold the feeling for few minutes, and make the physical movement required to laugh or smile, and then think and come up with fair attributes. Hold the reason for 5 minutes.
   Situation: Imagine a situation in your life where you often become angry and think how you would calm yourself by analyzing the fair attributes of that situation.

In a second level, the activity module 200 may present another list of tasks in a sequence.

Screen 1 shows the tasks as follows:
You might come across situations, where are going to restaurant and the service person there takes time to get your order. Then calm yourself by breathing deeply for 5 minutes and analyze the fair attributes (why the server is taking long time to serve you).

### Fair Attributes:

The restaurant is crowded.
You might come across situations, where you are going to travel by public transport and find that the ticket counter person is taking long time to issue the tickets. Then calm yourself by breathing deeply for 5 minutes and analyze the fair attributes (why the counter person is taking long time to issue you the tickets) and write them down.
You might come across a situation, where someone criticizes you. Then calm yourself, move out of that place and then analyze the fair attributes why the person might have criticized you, and then write them down objectively.
You might come across situation where you are waiting for your turn in a queue in food joint. Instead of getting angry, calm yourself by counting backwards from 10 slowly and analyze the fair attributes (what could be the reasons the queue is taking longer time to move) and write them down.
You might come across a situation, where you are waiting for someone is taking long time to reach and you have been waiting for a long time. Instead of getting angry, calm yourself by walking over to a window where there is sunlight and analyze the fair attributes (What could have caused their delay in arrival) and write them down.

### Screen 2:

By the end of the day, please write down your feelings and how you have coped successfully will each provoking situations. Also mention the fair attributes of the situation what you encountered during the day. A box may be provided to the individual to provide his response.

In a third level, the activity module 200 may present another list of tasks in a sequence.

Screen 1 shows the tasks as follows:
If you are a person who tends to get angry often while being hungry, try to avoid these situations, by eating on time and start performing relaxation techniques.
If any situation can provoke you, identify your physical changes that happen, try to calm yourself down by moving out of that situations immediately and try to analyze the fair attributes of the situations.
When you are traveling in a city where there is constant traffic troubles, try to calm yourself by listening to soothing music during your journey.
When someone is trying to interrupt you, instead of getting angry, assertively inform the person that you are working would like to get back to him/her later.
When someone is making you wait, try to analyze the why the person is delaying.

### Screen 2:

By the end of the day, please write down your feelings and how you have coped successfully with each provoking situations. Also mention the fair attributes of the situation what you encountered during the day. A box may be provided to the individual to provide his response.

In a fourth level, the activity module 200 may present another list of tasks in a sequence.

### Screen 1:

Meet the person who irritates you the most and try to keep your calm by analyzing the perception of the person. Try to analyze the fair attributes why that person reacts that way.

### Screen 2:

By the end of the day, please write down your feelings and how you have coped successfully with each provoking situations. Also mention the fair attributes of the situation what you encountered during the day. A box may be provided to the individual to provide his response.

The real world activity 220b may be presented to the user after the user has at least participated in one or more games (204c and/or 204d) to a predefined extent that attempts to rewire 225 the second defective brain wiring 216b. The real world activity 220b may be designed rewire the brain to increase the ability to weigh between fair and unfair attributes of a situation 225. Instructions 227b may include recommendation that the action has to be repeated for a predefined number of times every day and rules 229b may state that each level has to be repeated for a predefined number of times, before the next level unlocks.

In a first level, the activity module 200 may provide instructions 227h to choose a quiet place to sit in and close the eyes and imagine in detail what is shown. The activity module 200 may present a list of tasks in a sequence

Screen 1 shows the tasks as follows:
Imagine someone/some situation that provokes your anger, hold the feeling for few minutes, and visualize immediately a huge pipe is opened and the water is rushing from it. You are trying to close it successfully. Later, come up with fair attributes and unfair attributes of the situation. Think of an appropriate response and write it down.
Example
   Situation: You attended an interview that you did not get through.
Fair Attributes:
This job is just not cut out for me.
So many people in their lives have failed in interviews.
It was an opportunity to experience the interview process.
Unfair Attributes:
The interviewer was biased.
The interview process is to blamed.
The receptionist gave wrong direction, because of which you got little delayed

### Appropriate Response:

Remaining in calm and poised will help focus to the future interview:
Imagine someone/some situation provoke your anger, hold the feeling for few minutes, and try to do 10 sit up. Before you perform take a deep breath and then do a sit up. Do this for 10 times.
Later, come up with fair attributes and unfair attributes of the situation. Think of an appropriate response and write it down.
Imagine someone/some situation provoke your anger, hold the feeling for few minutes, and think about your destructive expression of your anger and replace with constructive expression.
Imagine someone/some situation provoke your anger, hold the feeling for few minutes, and imagine you telling them that you will listen to them later and immediately try to change the environment.

### Screen 2:

Your anger reflex should diminish another degree each time you do imagining.

In a second level, the activity module 200 may present a list of tasks in a sequence. Each time an action is carried out, the fair and unfair attributes of the situations are to be analyzed and then a rational and balanced conclusion has to be made.

Screen 1 shows the tasks as follows:
When you are busy working on something, and someone interrupts you, refrain from getting angry, try to calm yourself by taking 5 minutes break by exposing yourself to the sunlight.
Become mindful. Watch our thoughts and learn to separate your thoughts from your own identity. Your thoughts are not you.
When something/someone provokes your anger, stop yourself before you say something you'll soon regret, hold off for just a few moments, collect our thoughts by analyzing the both your perspective, then once you are calm, go to that person express your anger assertively,
When something/someone provokes your anger, stop yourself before you say something you'll soon regret, hold off for just a few moments, move out of that situation and perform sit up by simultaneously breathing deeply. Carry out this action 10 times.
When something/someone provokes your anger, stop yourself before you say something you will soon regret, hold yourself for some moments, try to calm yourself and write down what are the lair and unfair attributes of the of the situation.

### Screen 2:

Write the situation that you encountered at the end of the day, how you successfully calmed yourself and write the fair and unfair attributes of the situation. A box may be provided to the individual to provide his response.

In a third level, the activity module 200 may present a list of tasks in a sequence.

Screen 1 shows the tasks as follows:
When you are having an argument with your partner/friend/sibling, try to analyze the fair and unfair attributes of the argument and try make a judgment.
When something/someone provokes your anger, stop yourself before you say something you'll soon regret, hold off for just a few moments, collect your thoughts, allow others involved in the situation to do the same and try collecting their side of perspective.
When something/someone provokes your anger, stop yourself before you say something you'll soon regret, hold off for just a few moments, collect your thoughts, try to express your anger in a creative way either by painting, learning martial arts etc.
When someone does not do something you had asked them to do, try to understand why that person could not have been able to complete the task and
When someone make fun of you, try to take it in the face value by only analyzing fair attributes of the situation with the unfair attributes.

### Screen 2:

Write the situation that you encountered at the end of the day, how you successfully calmed yourself and write the fair and unfair attributes of the situation. A box may be provided to the individual to provide his response.

In a fourth level, instructions may be provided to the individual to mentor another individual with anger issues (and who wants to change) and guide them on the skills that you have learned. This person could be your friend/family member or colleague.

In an embodiment, the action database 206 further includes plurality of physical actions, mind actions and dietary actions, one or more of which may be presented to a user who is trying to overcome anger 214a.

Example of physical actions includes one or more physical activities, such as exercises and physical postures, among others. Example of mind actions may include meditation. Example of dietary actions may include adding specific food to one's diet or deleting specific food from the diet. As a further example, drinking black tea may be a dietary action and cutting down on rice consumption may be another dietary action.

### Logging Module 208

In an embodiment, the logging module 208 enables logging of the activities (ex: games, real world activities and other actions) performed by the user. The logging module 208 may record information corresponding to the activities performed by the user. The information may include one or more of, time of performance, date of performance, quantum of performance, success or failure consequent to an attempt to perform, and extent of success in performing the action, among others. The user may provide input that an activity has been performed. Such input may be used to log the activity in the logging module 208. Alternatively, the logging module 208 may be configured to automatically log the performance of an activity upon detection that the activity has been performed.

### Assessment Module 210

The assessment module 210 of the activity module 200 is configured to determine the performance of a user in each activity the user performs. The assessment module 210 may determine the user's score for each game and at each level in a came based on the log available in the logging module 208. Further, the assessment module 210 analyzes performance of the user in the real world activities and determines scores of each level of real world activity based on the log that is present in the logging module 208. The assessment module 210 may also determine status of the one or more behavioral trait 214 or the brain wiring causing the trait, after engaging in the activities, based on performance of activities suggested by the activity module 200.

### User Interface Module 212

The user interface module 212 may be configured to receive input from the user and display content to the user. The content displayed, can be, as an example, games, images, instructions, rules, information, haptic feedback and sound, among others.

### V. Method for Overcoming Anger

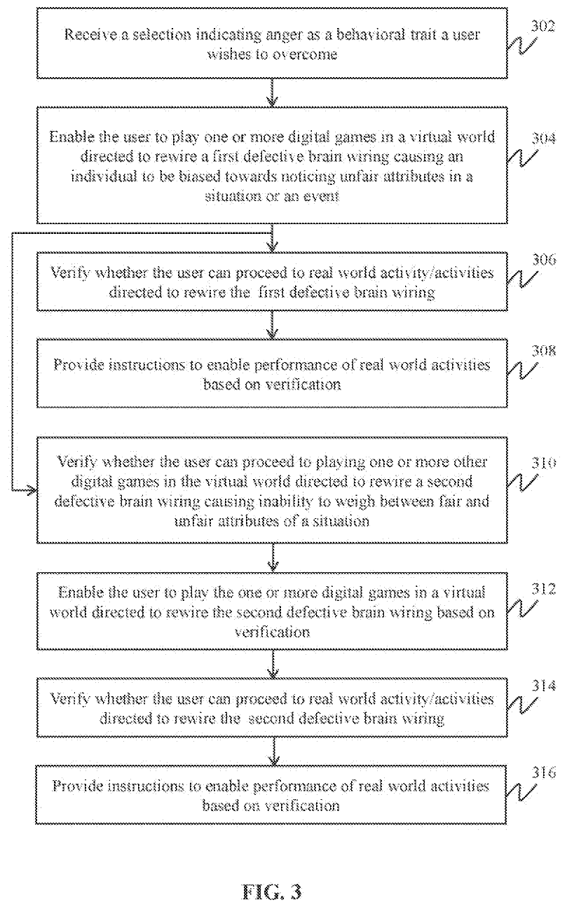

Now referring more particularly to FIG. 3, a method is provided for overcoming anger 214a. A selection indicating anger 214a as a behavioral trait which a user wishes to overcome is received at step 302. At step 304, a user is enabled to play/participate in one or more digital games 204a and/or 204b, which is played in a virtual world. The digital games 204a and 204b are instrumental in rewiring 219 a first defective brain wiring 216a, whose symptom is exhibition of anger 214a. At step 306, verification is made to determine whether the user can proceed to perform real world activities 220a. In case it is determined that user can proceed, then at step 308, instructions are provided to the user to enable the user to perform appropriate real world activity/activities 220a. Preferably, the user is allowed to perform or instructed to begin performing appropriate real world activities 220a upon participating in one or all of the digital games among 204a and 204b at least to a predefined extent, which is determined based on the desired rewiring. The real world activity 220a is instrumental in rewiring 219 the first defective brain wiring 216a, whose symptom is exhibition of anger 214a.

At step 310, verification is made to determine whether the user can proceed to playing one or more other digital games 204c-204d in the virtual world directed to rewire a second defective brain wiring 216b. In case it is determined that user can proceed, then at step 312 the user is enabled to play/participate in at one or all of the digital games among 204c and/or 204d, which are played in a virtual world. The digital games 204c-204d are instrumental in rewiring 225 the second defective brain wiring 216b, whose symptom is exhibition of anger 214a. Preferably, the user is allowed to proceed to playing one or all of the other digital games 204c-204d upon participating in the game(s) instrumental in rewiring 219 the first defective wiring 216a at least to a predefined extent, which is determined based on the desired rewiring.

At step 314, verification is made to determine whether the user can proceed to perform real world activities 220b. In case it is determined that user can proceed, then at step 316, instructions are provided to the user to enable the user to perform appropriate real world activity/activities 220b. Preferably, the user is allowed to perform or instructed to begin performing appropriate real world activities 220b upon participating in one or all of the digital game(s) 204c-204d at least to a predefined extent, which is determined based on the desired rewiring. The real world activity 220b is instrumental in rewiring 225 the second defective brain wiring 216b, whose symptom is exhibition of anger 214a.

Referring to the step (302) of receiving a selection indicating anger 214a, the user may be provided an option to select a behavioral trait that he would like to overcome. The activity module 200 may provide or recommend an option to select at least one weak behavioral trait to work on. The user may select at least one behavioral trait 214 that the user thinks he needs to work on, from a list of behavioral traits that may be displayed to the user via the user interface module 212. Alternatively, the user may input answers in response to a set of questions displayed to the user, which may determine what type of weak behavioral trait 214 the user may have. Upon selection of anger 214a, the activity module 200 may display a list 216d of other behavioral traits that can be confused with anger 214a, so that the user can select a more appropriate behavioral trait 214, which he desires to work on. The activity module 200 may further display the information corresponding to the brain wirings (defect) 216a, 216b causing the behavioral trait 214. The first defective brain wiring 216a causes the individual to be biased towards noticing unfair attributes in a situation or an event. The second defective brain wiring 216b causes inability to weigh between fair and unfair attributes of a situation.

The activity module 200 may additionally display information about impact 216c of anger 214a to the user. In addition, the activity module 200 may display information corresponding to the brain rewiring 219, 225 that the activity module 200 is attempting to achieve to overcome anger 214a. Rewiring 219 of the first defective brain wiring 216a results in increased ability to identify the fair attributes of a situation. Rewiring 225 of the second defective brain wiring 216b results in increased ability to weigh between fair and unfair attributes of a situation.

Furthermore, the activity module 200 may display the information corresponding to the causes (genetic or environmental) that resulted in the defective brain wirings (defect) 216a, 216b.

Referring to the step (304) or enabling the user to play at one or more digital games in the virtual world directed to rewire the first defective brain wiring 216a, the activity module 200 may select one or more digital games based on the capability of the digital games to achieve the desired brain rewiring 219.

The activity module 200 may provide a choice (one or more games) of games, from which the user may select. The activity module 200 may even indicate the effectiveness of each of the games in achieving the desired brain rewiring 219 or an aspect of rewiring.

In an embodiment, the activity module 200 is configured to recommend a plurality of games 204, wherein the recommendation indicates each of the games' 204 effectiveness to achieve the desired brain rewiring 219 or an aspect of rewiring.

The activity module 200 allows the user to play the selected game. The logging module 208 may log the performance of the user. The assessment module 210 may determine the performance scores using the log in the logging module 208. Further, based on the respective rules 223, the user is either allowed or denied access to other levels of the game 204.

Referring to step 306, the activity module 200 verifies whether the user can proceed to perform real world activities 220. Preferably, the activity module 200 recommends real world activities 220 after the user has performed to a desired extent in the digital game(s) 204. Desired extent of performance can be, as an example, levels completed, score reached and duration over which the game 204 was played.

In an embodiment, the activity module 200 recommends participating in real world activity 220a or part thereof after the user has performed to a desired extent in the digital game 204a. Likewise, the activity module 200 recommends participating in real world activity 220a or part thereof after the user has performed to a desired extent in the digital game 204b. Desired extent of performance can be, as an example, levels completed, score reached and duration over which the game(s) were played.

In an embodiment, the activity module 200 recommends participating in a level or part of real world, activity (ex: level of real world activity 220a) after the user has performed to a desired extent in a level of the digital game (ex: level of digital game 204a), wherein the level of the digital game prepares the user to participate in the level or part of the real world activity.

In an embodiment, the activity module 200 is configured to recommend a plurality of real world activities or sets of real world activities 220, wherein the recommendation indicates each of the real world activities' or sets of real world activities' 220 effectiveness to achieve the desired brain rewiring.

The logging module 208 may log the performance of the user. The assessment module 210 may determine the performance scores using the log in the logging module 208. Further, based on the rules 229, the user is either allowed or denied access to other levels of the real world activities or sets of real world activities.

Referring to step 310, the activity module 200 verifies whether the user can proceed to playing at least one or more other digital games in the virtual world directed to rewire the second defective brain wiring 216b causing inability to weigh between fair and unfair attributes of a situation. Rewiring the second defective brain wiring 216b results in ability to weigh between fair and unfair attributes of a situation.

In an embodiment, the activity module 200 recommends participating in digital games directed to rewire the second defective brain wiring 216b after the user has performed to a desired extent in the digital game(s) directed to rewire the first defective brain wiring 216a.

In an embodiment, the activity module 200 recommends participating in digital games directed to rewire the second defective brain wiring 216b after the user has performed to a desired extent in the digital garnet(s) and real world activity/activities directed to rewire the first defective brain wiring 216a.

The activity module 200, based on the verification, enables the user to play/participate in one or more other digital games 204c and 204d in the virtual world directed to rewire the second defective brain wiring.

In an embodiment, the activity module 200 recommends participating in real world activity 220b or part thereof after the user has performed to a desired extent in the digital game 204c. Likewise, the activity module 200 recommends participating in real world activity 220b or part thereof after the user has performed to a desired extent in the digital game 204d.

in another embodiment, the activity module 200 recommends participating in digital game 204b after the user has performed to a desired extent in a level of the digital game 204a. Similarly, the activity module 200 recommends participating in digital game 204c after the user has performed to a desired extent in a level of the digital game 204b. Likewise, the activity module 200 recommends participating in digital game 204d after the user has performed to a desired extent in a level of the digital game 204c.

Additionally, the activity module 200 may provide instruction to perform mind actions, physical actions and dietary actions.

Each of the physical and dietary actions may have effect on one or more hormones corresponding to one or more traits, among others. While executing the physical action the energy expended in the body is more compared to the energy expended while playing brain game. Similarly, the mind action may involve thinking about the specific trait in a way which may change the structure of the brain by changing the Axons, Receptors and/or the number of Neurons. The dietary action may include consuming specific foods which changes one or more hormone levels.

The real world, mind, physical and dietary activities are carried out in a real environment. Automated instructions are provided as to when and how the activities may have to be carried out. Further, there may be instructions that one or more of the real, world activities may have to be carried out simultaneously with the brain games and one or more of the activities may have to be carried out prior to or after engaging in brain game (digital games) and performing to a desired extent.

In an embodiment, upon selecting anger, the activity module 200 provides a scale operable b the user to select a position on the scale. The position may be indicative of the user's perception of his anger. Improvement in eradication or alleviation of anger resulting from the rewiring of the brain achieved by the performance of the activities (virtual games or real world activities) enabled by the activity module 200 is reflected on the scale. Degradation in eradication or alleviation of anger resulting from the degradation of the rewiring of the brain due to premature halt in performance of the activities (virtual games or real world activities) enabled by the activity module 200 is also reflected on the scale.

The games and the real world activities change the neural connections in the brain. When these activities are carried out repeatedly, new neural connections are formed (brain plasticity) but if one stops carrying out these activities prematurely then slowly the neural connections die.

The processes described above is described as sequence of steps, this was done solely for the sake of illustration. Accordingly, it is contemplated that some steps may be added, some steps may be omitted, the order of the steps may be re-arranged, or some steps may be performed simultaneously.

The example embodiments described herein may be implemented in an operating environment comprising software installed on a computer, in hardware, or in a combination of software and hardware.

Although embodiments have been described with reference to specific example embodiments, it will be evident that various modifications and changes may be made to these embodiments without departing from the broader spirit and scope of the system and method described herein. Accordingly, the specification and drawings are to be regarded in an illustrative rather than a restrictive sense

Many alterations and modifications of the present invention will no doubt become apparent to a person of ordinary skill in the art after having read the foregoing description. It is to be understood that the phraseology or terminology employed herein is for the purpose of description and not of limitation. It is to be understood that the description above contains many specifications, these should not be construed as limiting the scope of the invention but as merely providing illustrations of some of the personally preferred embodiments of this invention.

1-3. (canceled)

4. A system for improving personality traits, wherein the system comprises 1) a data store containing two or more sets of personality trait information about two or more personality traits, respectively, and 2) at least one processor configured to present at least one digital page containing content taken from the two or more sets of personality trait information, the at least one processor further configured to:
provide an option to a user for selecting at least one personality trait from the personality traits;
suggest actions including a physical action, a mind action, and a brain action, the suggested actions based on the personality trait selected by the user,
wherein the suggested actions are to cause an improvement in the selected personality trait of the user, in which the content presented on the at least one digital page, taken from a set of the two or more sets of personality trait information and corresponding to the personality trait selected by the user, facilitates performance by the user of the suggested mind action or the suggested brain action.

5. The system according to claim 4, wherein the at least one processor is further configured to:
suggest at least one alternate personality trait for selection based on the personality trait selected by the user; and
allow selection of at least one of the suggested alternate personality traits or proceed with the earlier selection.

6. The system according to claim 4, wherein the at least one processor is further configured to provide a scale operable by the user to select a position on the scale, wherein the position is indicative of the user's perception of his character corresponding to the selected personality trait.

7. The system according to claim 4, wherein the actions are suggested based on at least actions which have previously executed by the user.

8. The system according to claim 4, wherein the at least one processor is further configured to determine status of the selected personality trait by considering a user defined position of a status of the selected personality trait and predicted degradation of the improvement based on premature halting performance of the suggested actions.

9. The system according to claim 8, wherein the at least one processor is further configured to enable the user to alter the status of the selected personality trait from the status determined by the system; and
wherein the at least one processor is further configured to calibrate future determination of the status of at least the selected personality trait based on difference between the status determined by the system and the status altered by the user.

10. The system according to claim 8, wherein the deterioration of impact of an action among the actions performed by the user is based on a time elapsed since the action was performed.

11. An apparatus, comprising:
a data store containing one or more sets of personality trait information about one or more personality traits, respectively; and
a processor to present at least one digital page containing content taken from the one or more sets of personality trait information;
a memory storing instructions executable by the processor to assess a current level of a personality trait of a person, wherein the instructions, when executed by the processor, perform operations comprising:
identifying, from an input, information about an original assessment of a personality trait of the person, wherein the original assessment is indicative of an original level of the personality trait of the person;
suggesting actions including a physical action, a mind action, and a brain action, the suggested actions based on the personality trait of the person;
wherein the suggested actions are to cause an improvement in the personality trait of the person, in which content presented on the at least one digital page, taken from a set of the one or more sets of personality trait information and corresponding to the personality trait of the person, facilitates performance by the person of the suggested mind action or the suggested brain action;
deterministically modifying a value associated with the original assessment based on 1) information about which one(s) of the suggested actions are performed by the person and 2) predicted degradation of the improvement based on premature halting performance of the suggested actions; and
providing an updated assessment of the personality trait of the person based on said deterministically modifying.

12. The apparatus of claim 11, wherein the operations further include:
allowing a user to select one or more actions from the suggested actions; and
enforcing restriction on type of actions that the user can select at least based on effect of hormones on the personality trait and effect of mind on the personality trait.

13. The apparatus of claim 11, wherein the one or more personality traits comprise a plurality of personality traits, wherein the operations further comprise:
providing an option to a user for selecting at least one personality trait from the plurality of personality traits;
suggesting at least one alternate personality trait for selection based on the personality trait selected by the user; and
allowing selection of at least one of the suggested alternate personality traits or proceed with the earlier selection.

14. The system according to claim 11, is further configured to suggest at least one alternate action upon the user deferring at least one of the suggested actions.

15. A method comprising:
providing 1) a digital scale operable by a user to select a position on the digital scale, and 2) at least one digital page containing content taken from one or more sets of personality trait information about one or more personality traits, wherein the position on the digital scale is indicative of the user's perception of his character corresponding to an identified personality trait of the one or more personality traits;
suggesting actions including a physical action, a mind action, and a brain action, the suggested actions based on the identified personality trait;
wherein the suggested actions are to cause an improvement in the identified personality trait, in which the content presented on the at least one digital page, taken from a set of the one or more sets of personality trait information and corresponding to the identified personality trait, facilitates performance by the user of the suggested mind action or the suggested brain action;
determining a current status of the personality trait at least based on the user selected position on the scale and ones of the suggested actions that are performed by the user.

16. The method according to claim 15, further comprising enforcing restriction on type of actions that the user can select at least based on effect of hormones on the personality trait and effect of mind on the personality trait.

17. The method according to claim 15, wherein determining the current status of the identified personality trait further comprises considering deterioration of impact of one(s) of the suggested actions that are performed by the user.

18. The system of claim 4, the content on the presented at least one digital page includes information specifying:
a response pattern to external stimuli that predisposes individuals to the personality trait selected by the user; and
a target brain wiring that is opposite of the response;
the at least one processor further configured to:
provide a language based facts game to use the specified information and present, to the individual, a situation and a number of associated facts along with the situation, wherein to play the language based facts game the individual provides answers or selects answers by applying the opposite of the response pattern in an evaluation of the facts; and
carry out the language based facts game repeatedly to aid the user to change the strength of the neural connection, via brain plasticity from repeated utilization of the brain wiring in the performance of the combination of actions.

19. The apparatus of claim 11, the content on the presented at least one digital page includes information specifying:
a response pattern to external stimuli that predisposes individuals to the personality trait of the person; and
a target brain wiring that is opposite of the response;
the at least one processor further configured to:
provide a language based facts game to use the specified information and present, to the individual, a situation and a number of associated facts along with the situation, wherein to play the language based facts game the individual provides answers or selects answers by applying the opposite of the response pattern in an evaluation of the facts; and
carry out the language based facts game repeatedly to aid the person to change the strength of the neural connection, via brain plasticity from repeated utilization of the brain wiring in the performance of the combination of actions.

20. The apparatus of claim 19, wherein each set of prediction values includes at least one additional prediction value indicative of a predicted impact of actual use of the language based facts game; and
wherein the deterministically modifying the value associated with the original assessment is further based on information about monitored use of the language based facts game by the person.

21. The method of claim 15, the content on the presented at least one digital page includes information specifying:
a response pattern to external stimuli that predisposes individuals to the personality trait selected by the user; and
a target brain wiring that is opposite of the response;
wherein the method further comprises:
providing a language based facts game to use the specified information and present, to the individual, a situation and a number of associated facts along with the situation, wherein to play the language based facts game the individual provides answers or selects answers by applying the opposite of the response pattern in an evaluation of the facts;
wherein the suggested actions include playing the language based facts game repeatedly to aid the user to change the strength of the neural connection, via brain plasticity from repeated utilization of the brain wiring in the performance of the combination of actions; and
monitoring use of the language based facts game by the user, wherein determining the current status of the personality trait at least based on a result of the monitoring.
